(19) United States
(12) Plant Patent Application Publication
(10) Pub. No.: US 2016/0106017 P1
(43) Pub. Date: Apr. 14, 2016

(54) Phalaenopsis plant named 'TJ PURPLE QUEEN'

(50) ;

(71) Applicant: Chang-Lian Lin, San Jose, CA (US)

(72) Inventor: Yuan-Yang Liao, Tainan (TW)

(21) Appl. No.: 14/121,698

(22) Filed: Oct. 8, 2014

(51) Int. Cl. A01H 5/00

(52) U.S. Cl. PLT/311

(57) ABSTRACT

A new and distinct cultivar named ‘TJ PURPLE QUEEN’ is disclosed, characterized by unique flowers which are large white with real purple petals and compact plant habit. The new variety is a Phalaenopsis hybrid cultivar, and is normally used as an ornamental plant with long and sturdy inflorescences.

### FIELD OF THE INVENTION

### Bontanical Classification

Phalaenopsis hybrid

### Variety Denomination

TJ Purple queen

### BACKGROUND OF THE INVENTION

The present invention comprises a new and distinct Phalaenopsis hybrid Orchid cultivar, and hereinafter referred to by the cultivar name, TJ PURPLE QUEEN.

The new cultivar was discovered as part of a commercial breeding program, carried out in a commercial greenhouse in Tainan, Taiwan. The inventor, Yuan Yang Liao, a citizen of Taiwan, discovered the new variety as the result of the crossing of the female parent, the unpatented variety Phalaenopsis hybrid ‘Sogo Yukidian’ (not patented) with the male parent, the unpatented variety Phalaenopsis hybrid ‘I-Hsin Spotted Deer.’ (not patented) The crossing was performed in April 2006, the new variety was discovered in March 2006 by the inventor.

Asexual reproduction of the new cultivar ‘TJ PURPLE QUEEN’ by tissue culture was performed at a commercial laboratory in Tainan, Taiwan later of 2006. Since that time, many generations have been reproduced, and have shown that the unique features of this cultivar are stable and reproduced true to type through successive generations.

### SUMMARY OF THE INVENTION

The cultivar ‘TJ PURPLE QUEEN’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘TJ PURPLE QUEEN.’ These characteristics in combination distinguish ‘TJ PURPLE QUEEN’ as a new and distinct Phalaenopsis hybrid cultivar:

1. This plant is with shorter & standing leaves compared to those with the other plant which with the similar size of flowers.

2. Unique pure & clear white with real purple bloom coloration.

3. Rough sepal and petal texture.

4. PARENT COMPARISON:

Plants of, the new cultivar ‘TJ PURPLE QUEEN’ are similar to plants of the seed parent variety Phalaenopsis hybrid ‘Sogo Yukidian’ (not patented) in some horticultural characteristics. However, plants of the new cultivar ‘TJ PURPLE QUEEN’ inherited the rough sepals and petals from this seed parent. Additionally, plants of ‘TJ PURPLE QUEEN’ produce white flowers (RHS N999D) with real purple spots (RHS N81B), whereas ‘Sogo Yukidian” with only the big white flowers (RHS 155C). Plants of the new cultivar ‘TJ PURPLE QUEEN’ are more similar to plants of the pollen parent variety, Phalaenopsis hybrid ‘I-Hsin Spotted Deer’ (not patented) in most horticultural characteristics. However, plants of the new cultivar ‘TJ PURPLE QUEEN’ produce white flowers with purple spots which looks like from Chinese classic painting, whereas ‘I-Hsin Spotted Deer’ produces white (RHS N999D) colored flowers with brown spots (RHS 83B).

### BRIEF DESCRIPTION OF THE DRAWINGS

Colors in the photographs may appear different from the color values that appear in the detailed botanical description which more accurately describe the new cultivar.

The photograph was taken using conventional techniques and although colors may appear different from actual colors due to light reflectance it is as accurate as possible by conventional photographic techniques.

FIG. 1 is a side view of a plant of TJ Purple queen flowering in the commercial 9 cm pot.

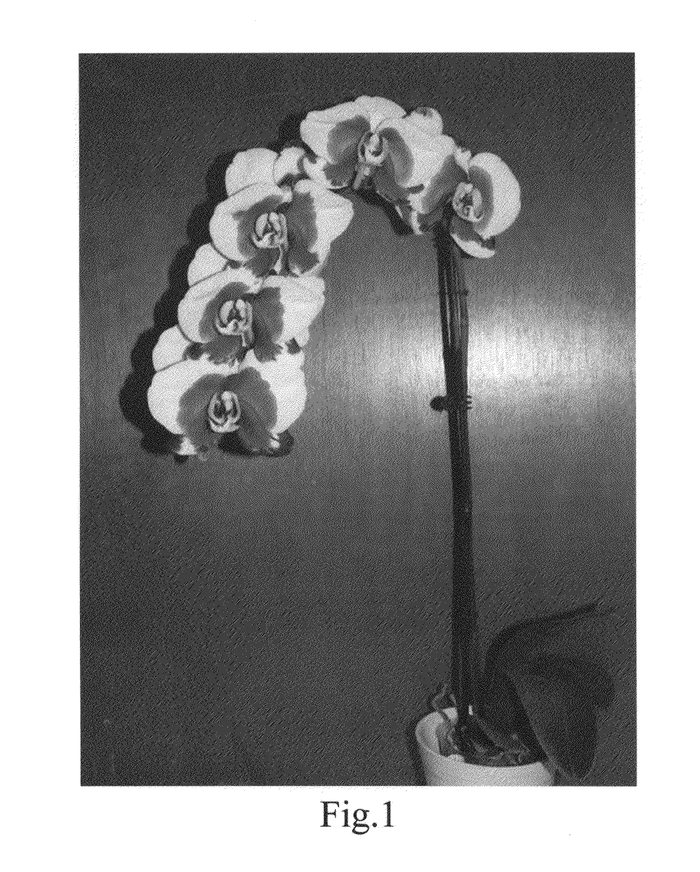

FIG. 2 is a side view of a plant of TJ Purple queen flowering in the commercial 9 cm pot with all black background.

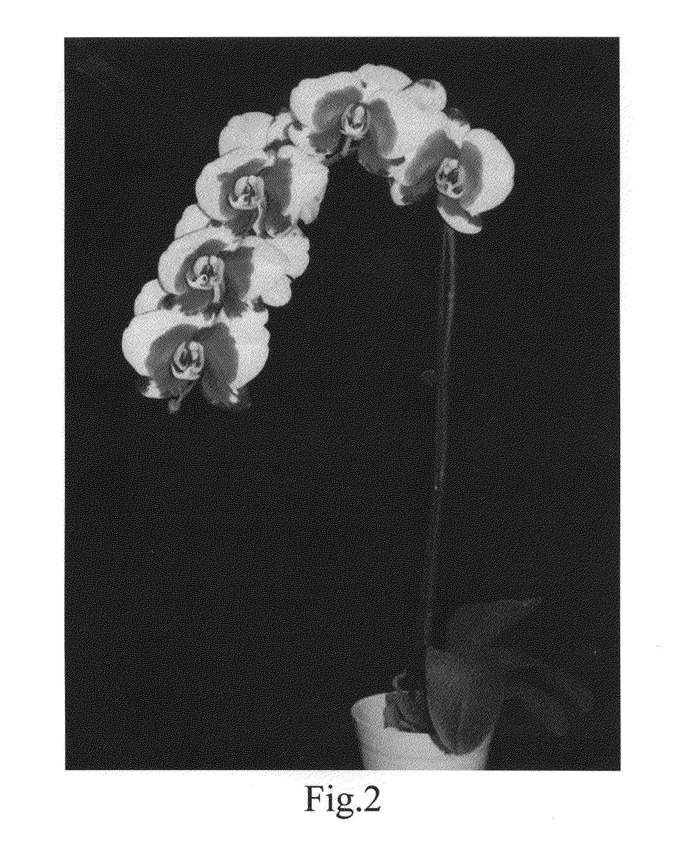

FIG. 3 is a side view of a plant of TJ Purple queen showing the characteristics of the flowers.

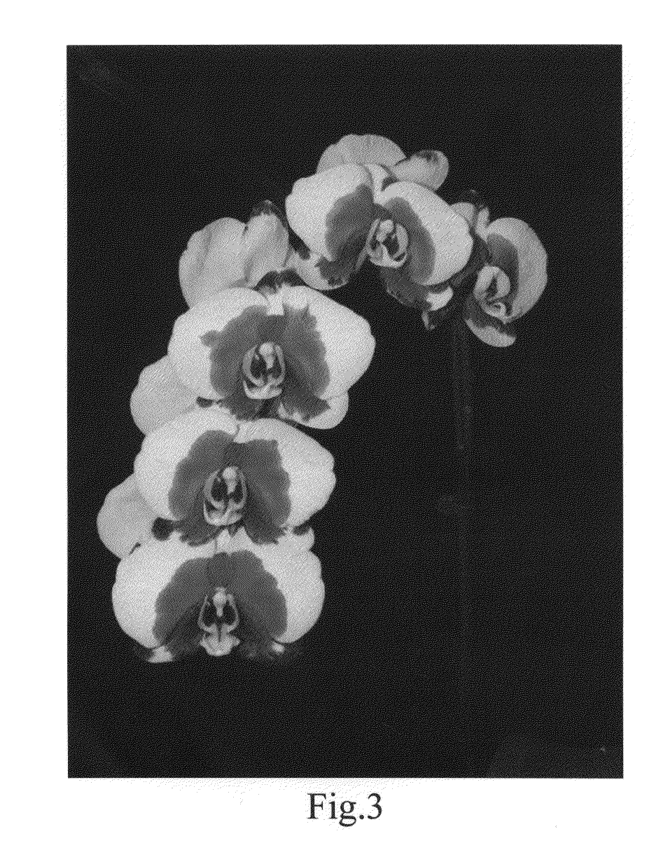

FIG. 4 is a close-up view showing the characteristics of the flower.

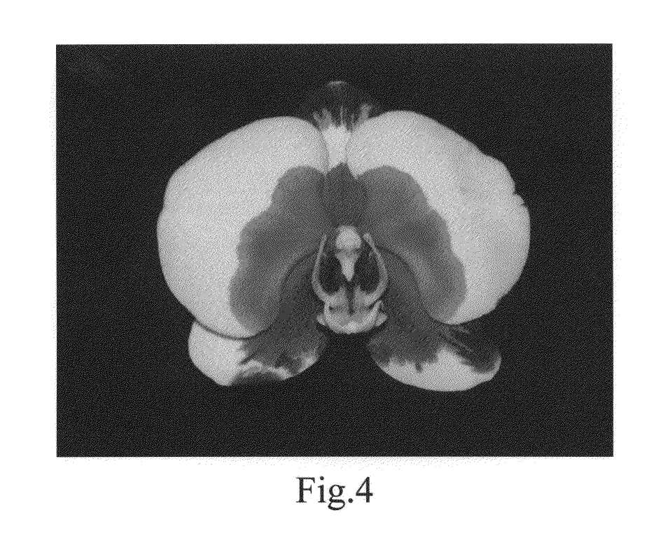

### DETAILED DESCRIPTION OF THE PREFERRED EMBODIMENTS

The following observations and measurements describe ‘TJ PURPLE QUEEN’ plants grown in a commercial greenhouse in Tainan, Taiwan. Temperature ranged from 23° C. to 26° C. day and from 16° C. to 20° C. night. Light levels ranged from 10,000 lux as a young plant to approximately 20,000 lux for a flowering plant. Plants described began flowering in the end of winter. Measurements and numerical values represent averages of typical flowering types. No chemical or photoperiodic treatments were given.
Parentage:
   Seed.—Phalaenopsis hybrid ‘Sogo Yukidian’ (not patented).
   Pollen.—Phalaenopsis hybrid ‘I-Hsin Spotted Deer’ (not patented).
Propagation:
   Type of propagation typically used.—Tissue culture.
   Time to initiate roots.—About 40 days at approximately 28-32° C.
   Time to produce a rooted cutting/liner.—About 5-6 months after cloning at approximately 28-35° C.
   Root description.—Thick (typical for Phalaenopsis) and rounded, average diameter: 5 mm, colored near RHS Yellow Green 145A.
Plant description:
   Plant shape.—Two-ranked leaves affixed to a short central stem (monopodial growth). Single flowers arranged on upright and sturdy flowering rancemes.
   Plant height.—Soil level to top of foliar plane. — About 20 to 25 cm.
   Plant height.—Soil level to top of inflorescences. — About 55 to 65 cm.
   Plant diameter.—About 25 to 35 cm.
Flowers per stem: Approximately 8 to 10.
Foliage description:
   Leaf arrangement.—Distichous.
   Quantity per plant.—About 6 to 7.
   Length.—About 20 to 25 cm.
   Width.—About 6 to 7 cm.
   Shape.—Obovate.
   Apex.—Unequal acute.
   Base.—Cuneate Sheath length: Average 2.1 cm Sheath width: Average 2.6 cm. Sheath.
   Margin.—Entire.
   Aspect.—Mostly flat and folded upward from the midrib.
   Texture, upper and lower surfaces.—Smooth, glossy, slightly furrowed
   Venation.—parallel.
   Color.—Upper surface — near RHS green N137A.
   Color.—Lower surface — near RHS green N137.
Flower description:
   Inflorescence type.—raceme.
   Flower type.—single zygomorphic flowers arranged in racemes, flowers roughly pentagonal in shape.
   Flower arrangement.—compound alternate.
   Flowering stems.—smooth moderately glossy.
   Flowering habit.—plants freely flowering; plants typically produce 0 branched flowering stems with at least 7 to 12 flowers each.
   Fragrance.—unscented.
   Natural flowering season.—from February to June in south of Taiwan.
   Post-production longevity.—Plants of the ‘TJ Purple queen’ maintain good leaf and flower substance for about 2 to 3 months on the plant under interior environmental conditions.
   Inflorescence length.—about 30 to 40 cm.
   Inflorescence diameter.—about 15 to 16 cm.
   Flower diameter.—about 9.5 to 11.5 cm.
   Flower depth.—about 4 cm.
Flower bud:
   Flower bud shape.—bud apex is orbicular bud base is ovate.
   Flower bud length.—about 3 cm.
   Flower bud diameter.—about 2 cm.
   Flower bud color.—yellow-green (RHS145B).
Petals:
   Number of petals.—2 per flower.
   Length.—about 6.8 cm.
   Diameter.—about 6.5 cm.
   Shape.—reniform.
   Apex.—rounded.
   Base.—broad attenuated.
   Margin.—entire.
   Texture, upper and lower surfaces.—upper surface dull somewhat velvety lower surface: rough, somewhat velvety.
   Petal main color.—white (RHS N999D).
   Petal pattern color.—light mauve (RHS N81B).
Labellum:
   Length.—about 2.9 cm.
   Diameter.—about 2.3 cm.
   Shape.—the labellum is three-lobed with two prominent callosities at the central junction of the lateral lobes and lobes and base of the midlobe.
   Base color of the apical lobe.—brilliant orangish yellow (RHS 21B).
   Tip color of the apical lobe.—white(RHS N999D).
Sepals:
   Quantity.—3 per flower.
   Dorsal sepal length.—about 4.5 cm.
   Lateral sepal length.—about 4.5 cm.
   Dorsal sepal diameter.—about 3.4 cm.
   Lateral sepal diameter.—about 3 cm.
   Shape.—ovate to broad elliptic.
   Apex.—obtuse to broad bluntly acute.
   Base.—broad cuneate.
   Margin.—entire.
   Texture, upper and lower surfaces.—smooth, dull, somewhat velvety.
   Dorsal sepal main color.—white (RHS N999D).
   Dorsal sepal pattern color.—light mauve (RHS N81B).
   Lateral sepal main color.—white (RHS N999D).
   Lateral sepal pattern color.—light mauve (RHS N81B).
Peduncles:
   Length.—about 78 to 82 cm.
   Diameter.—about 0.8 mm.
   Aspect.—40° angle to rosette.
   Strength.—strong.
   Texture.—smooth, not very glossy.
   Color.—in between RHS Dark Green 137A and RHS Dark Green 137A.
Pedicels:
   Length.—about 4 cm.
   Diameter.—about 0.4 mm.
   Aspect.—about 70° from vertical.
   Strength.—moderately strong.
   Texture.—smooth moderately glossy.
   Color.—yellow(RHS 2D).
   Color towards the base.—green (RHS 141D).
Reproductive organs:
Column:
   Length.—about 1 cm.
   Diameter.—about 6 mm.
   Color.—white (RHS N999D).
   Color towards to apex.—white (RHS N999D).
Pollinia:
   Quantity of pollen masses.—2.
   Diameter.—about 1.8 mm.
   Color.—yellow-orange (RHS 22A).
Ovary:
   Length.—about 5 mm.
   Diameter.—about 2.5 mm.
   Color.—white (RHS N999D).
Root: It takes 10 to 20 days for plants growing in tissue culture to initiate roots.
   Diameter.—about 5 mm.
   Color.—yellow green (RHS 145A).
Disease/pest resistance: Strong disease resistance, but no specific resistance or susceptibility to pests has been observed.
Temperature tolerance: The plant can be tolerant to temperature from 16-36° C. . The flower can be tolerant from 20-28° C.

What is claimed is:

1. A new and distinct cultivar of Phalaenopsis hybrid orchid plant named ‘TJ purple queen’ as illustrated and described.
